(19) United States
(12) Patent Application Publication
(10) Pub. No.: US 2005/0065584 A1
(43) Pub. Date: Mar. 24, 2005

(54) System and method for cooling internal tissue

(76) Inventors: Jonathan D. Schiff, Andover, MA (US); Timothy W. Robinson, Sandown, NH (US); Todd S. Saunders, Etna, NH (US); Matt J. Murphy, Braintree, MA (US); Samuel J. Akins, Boxborough, MA (US)

Correspondence Address: John Christopher;Christopher & Weisberg, P.A., Suite 2040, 200 East Las Olas Boulevard, Fort Lauderdale, FL, 33301, US

(21) Appl. No.: 10/936,076

(22) Filed: Sep. 8, 2004

Related U.S. Application Data
(60) Provisional application No. 60/501,313, filed on Sep. 9, 2003.

(51) Int. Cl. A61F 7/00; A61F 7/12

(52) U.S. Cl. 607/105; 607/113

(57) ABSTRACT

A system, device and method for thermally affecting tissue are provided. The system includes a pump/controller unit having a pump for pumping a thermally conductive fluid through the system, a fluid chiller for thermally treating the conductive fluid, a controller circuitry for measuring and controlling the temperature of the conductive fluid, and a fluid circulation path having an extension tubing set for circulating the thermally conductive fluid, a thermal application device having at least one flow passage that is in fluid communication with the tubing set, a thermal exchanger element in fluid communication with the tubing set that interfaces with the fluid chiller, where the tubing set operably interfaces with a pump to enable the pump to circulate a thermally conductive fluid through a fluid circulation path. The fluid circulation path may have various sensors providing temperature and pressure readings to the pump/controller unit.

### CROSS-REFERENCE TO RELATED APPLICATION

This application is related to and claims priority to U.S. Provisional Patent Application Ser. No. 60/501,313, filed Sep. 9, 2003, entitled SYSTEM AND METHOD FOR COOLING INTERNAL TISSUE, the entirety of which is incorporated herein by reference.

### STATEMENT REGARDING FEDERALLY SPONSORED RESEARCH OR DEVELOPMENT

Not applicable

### FIELD OF THE INVENTION

The present invention relates to a system, device and method for thermal treatment of body tissue of a patient, and in particular for neurosurgical environments to treat brain and cranial tissue of a patient using an electronic controller and a fluid circulation path.

### BACKGROUND OF THE INVENTION

Researchers and physicians have long recognized the consequences of reduction of body temperature in mammals, including induction of stupor, tissue damage, and death. Application of freezing and near freezing temperatures to selected tissue is commonly employed to preserve tissue and cell (e.g. sperm banks); and application of extreme cold (far below freezing) is effective for tissue ablation. However, localized cooling (not freezing) of tissue has generally been limited to the placement of an “ice-pack” or a “cold compress” on injured or inflamed tissue to reduce swelling and the pain associated therewith. Localized cooling of internal organs, such as the brain, has remained in large part unexplored.

For example, “brain cooling” has been induced by cooling the blood supply to the brain for certain therapies. However, as the effects of the cool blood cannot be easily localized, there is a systemic temperature reduction throughout the body that can lead to cardiac arrhythmia, immune suppression and coagulopathies.

Although attempts have been made to localize cooling of the brain with wholly external devices, such as cooling helmets or neck collars, there are disadvantages associated with external cooling to affect internal tissue. For example, external methods do not provide adequate resolution for selective tissue cooling, and some of the same disadvantages that are associated with systemic cooling can occur when using external cooling devices.

It is therefore desirable to obtain improved systems, devices and methods that allow for localized brain cooling without the disadvantages of the known systemic and external devices and techniques.

### SUMMARY OF THE INVENTION

The present invention advantageously provides a system, device and method for thermally affecting tissue of a patient. According to an aspect of the present invention, a system for thermally affecting tissue of a patient is provided in which a pump/controller unit includes a pump for pumping a thermally conductive fluid through the system, a fluid chiller for thermally treating the conductive fluid, a controller circuit for measuring and controlling the temperature of the conductive fluid, and a fluid circulation path in which the fluid circulation path includes an extension tubing set for circulating the thermally conductive fluid and interfacing with the pump, a thermal application device, and a thermal exchanger element that interfaces with the fluid chiller. The system may also include an optional fluid reservoir with corresponding control valves.

According to another aspect of the present invention, a system for thermally affecting tissue of a patient is provided in which a pump/controller unit includes a pump for pumping a thermally conductive fluid through the system, a fluid chiller for thermally treating the conductive fluid, a controller circuit for measuring and controlling the temperature of the conductive fluid, and a fluid circulation path having an extension tubing set for circulating the thermally conductive fluid and interfacing with the pump, a set of control valves providing the capability to selectively operate the system in a closed or open loop configuration, an optional fluid reservoir, a thermal application device, and a thermal exchanger element that interfaces with the fluid chiller.

According to yet another aspect of the present invention, a method for thermally affecting a tissue treatment site in the body of a patient is provided in which a medical device is selected to thermally affect the tissue treatment site. The medical device includes an expandable body defining a tissue contact area. An opening is created in the patient's body. The expandable body is inserted into the opening such that the tissue contact area is in thermal communication with the tissue treatment site. A thermally transmissive fluid is infused into the expandable body.

According to yet another aspect of the present invention, a medical device for thermally affecting tissue is provided in which a cap includes a bottom region and a top region and an expandable body, which includes a wall defining an interior volume and a tissue contact surface. The cap top region has a fluid inlet conduit and a fluid outlet conduit. The expandable body is coupled to the cap bottom region such that the interior volume is in fluid communication with the fluid inlet and the fluid outlet. The medical device may also include one or more sensors for measuring temperature or pressure of a patient's tissue or of thermally transmissive fluid.

According to still yet another aspect of the present invention, a method for thermally affecting a tissue treatment site in the body of a patient is provided in which a medical device is selected to thermally affect the tissue treatment site. The medical device includes an expandable body defining a tissue contact area. An opening is created in the patient's body. The expandable body is inserted into the opening such that the tissue contact area is in thermal communication with the tissue treatment site. A thermally transmissive fluid is infused into the expandable body.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a top level block diagram of an exemplary embodiment of the system according to the present invention;

FIG. 2 is a block diagram of an exemplary embodiment of the system according to the present invention;

FIG. 3 is a block diagram of the Pump/Controller Unit of the exemplary system of FIG. 2;

FIG. 4 is another exemplary embodiment of the system according to the present invention;

FIG. 5 is a perspective view of the tubing set sensors in communication with the thermal application device;

FIG. 6 is a perspective view of an exemplary embodiment of a device constructed in accordance with the principles of the present invention;

FIG. 7 is a bottom view of the cap of the device shown in FIG. 6;

FIG. 8 is a side perspective view of the cap of the device illustrating an exemplary routing of a sensor wire to a sensor;

FIG. 9A is side perspective view of a sensor inside but unattached to the expandable body of an exemplary device;

FIG. 9B is side perspective view of a sensor inside and attached to the expandable body of an exemplary device;

FIG. 9C is side perspective view of two sensors inside and attached to the expandable body of an exemplary device;

FIG. 9D is side perspective view of a sensor routed outside the expandable body of the device and attached proximate to tip of the expandable body of an exemplary device;

FIG. 9E is side perspective view of a sensor enclosed in a flexible tube that insulates it from the cooling fluid of an exemplary device; and

FIG. 10 is a perspective view of an exemplary embodiment of a device deployed to contact a tissue treatment site of the brain tissue in accordance with the principles of the present invention.

### DETAILED DESCRIPTION OF THE INVENTION

The present invention provides a system, device and method for the application or removal of thermal energy to or from a localized region of a body tissue.

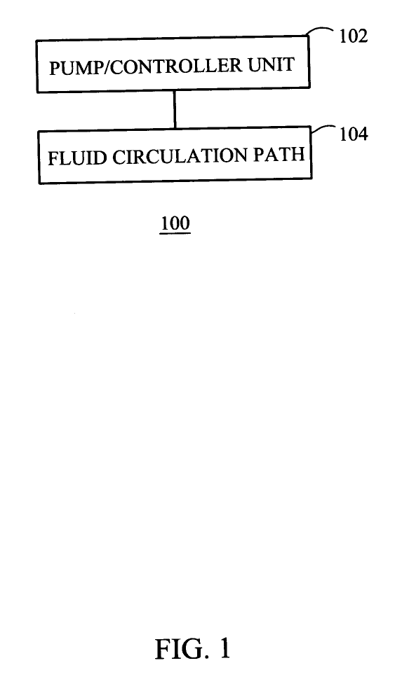

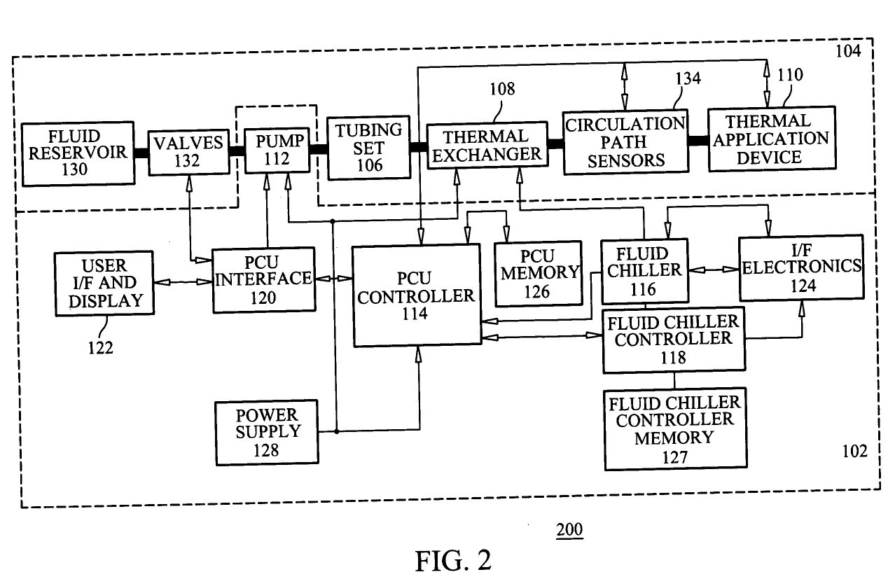

Referring now to the drawing figures in which like reference designators refer to like elements, there is shown in FIG. 1, a top level block diagram of an exemplary embodiment of the system according to the present invention and designated generally as system 100. The system 100 includes a pump/controller unit (PCU) 102 and a circulation path 104. In FIG. 2, a system 200 is shown, which provides further details of the system 100 of FIG. 1. The system 200 includes the pump/controller unit 102 and the circulation path 104. The fluid circulation path 104 includes an extension tubing set 106, a thermal exchanger element 108, a thermal application device 110 (referred to herein as the “pad”) for contact with the tissue to be treated, a set of circulation path sensors 134, a set of optional valves 132 and an optional fluid reservoir 130.

The extension tubing set 106 is made of a suitable material, for example, PVC or urethane, and is coupled to the thermal exchanger element 108 and the thermal application device 110. The extension tubing set 106 preferably uses tubing that is ⅛″ inner diameter by ¼″ outer diameter, but also may have tubing of various diameters. Examples of the thermal application devices 110 include various strips, pads, “buttons” and other suitable configurations that are arranged to contact internal tissue for treatment. Such devices will be described in more detail below.

The fluid circulation path 104 includes a thermal exchanger element 108 to cool or heat the thermally conductive fluid in the circulation path 104. The thermal exchanger element 108 contacts or engages a fluid chiller 116 in the pump/controller unit 102 in such manner as to allow the transfer of heat or cold from the thermal exchanger element 108 to the fluid chiller 116. According to one embodiment, the thermal exchanger element 108 includes a body and an outer face having a thin membrane covering a serpentine fluid path. When the outer face is applied to the source of cold, for example, the fluid chiller 116, and fluid is pumped through the serpentine fluid path, heat is removed from the fluid via the thin membrane thereby cooling the fluid. This arrangement advantageously allows the thermally conductive fluid to be cooled or heated while preserving its sterility. By way of non-limiting example, the body of the thermal exchanger element 108 can be made of plastic, such as polyethylene, with the thin membrane also being made of plastic, such as a 0.003″ thick polyester/polyethylene sheet. Other membrane materials may be used, for example, aluminum, copper, platinum, gold, palladium, and other “designer” metals, provided that biocompatibility with the tissue to be contacted is maintained. Additionally, the heat exchanger element 108 may be designed to have its inlet path and outlet path on the same side or on opposite sides.

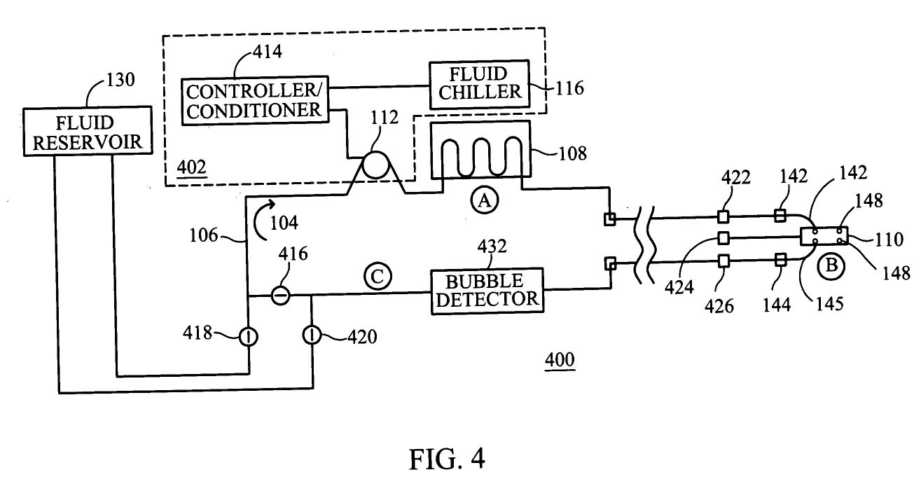

An optional set of valves 132 may be coupled to an optional fluid reservoir 130 and the pump 112. The optional set of valves 132 and optional fluid reservoir 130 will be described in further detail in the section below detailing FIG. 4.

The pump/controller unit 102 controls the flow and temperature of the thermally conductive fluid circulating through the circulation path 106. The pump/controller unit 102 may be placed in a housing for portable distribution, or alternatively may reside on a cart or shelf. It is contemplated that the pump/controller unit 102 can be arranged to control one or more circulation paths. The pump/controller unit 102 delivers thermally conductive fluid, such as chilled saline, through the extension tubing set 106 to the pad 110 at temperatures cold enough to allow the surface of the pad in contact with the patient's tissue to provide the desired benefit. For example the temperature of the exterior surface of the pad 110 in contact with the patient's brain can be maintained at 15° C.,±1° C. Among other things, the pump/controller unit 102 monitors the temperature of the thermally conductive fluid at multiple points in the circulation path, provides cooling, monitoring and pumping functions, and provides the user interface for the system 200.

The pump/controller unit 102 includes a pump 112, a PCU controller 114, a fluid chiller 116, a fluid chiller controller 118, a power supply 128, and a user interface 122. The pump/controller unit 102 may further include an optional PCU interface electronics 120, an optional PCU memory 126, an optional fluid chiller interface electronics 124 and an optional fluid chiller memory 127. Although the pump 112 is preferably a peristaltic pump adapted to use tubing with 1/16″ thick walls, other types of positive displacement pumps, such as but not limited to piston pumps and roller pumps, centrifugal pumps, or any other pump that can maintain the sterility of the thermally conductive fluid in the fluid path 104 can be used. A peristaltic pump is preferred in the present implementation because it can pump coolant without directly contacting the coolant, by simply squeezing a tube through which the conductive fluid flows.

The extension tubing set 106 may have a section or sections of tubing with sufficient elasticity and robustness to interface directly with the pump 112. In one embodiment, the pump 112 is set internally to run at a constant speed once it is activated. Alternatively, it is also contemplated that the speed of the pump 112 can be adjusted, as necessary, to achieve a desired fluid flow rate. Additionally, the rotation of the pump 112 can be reversed under user control to rapidly evacuate the pad 110 thereby reducing the pad's volume, and thus simplifying pad 110 extraction from the patient. The evacuation of the pad 110 may be accomplished through the use of additional check valves or the operation of the optional valves 132.

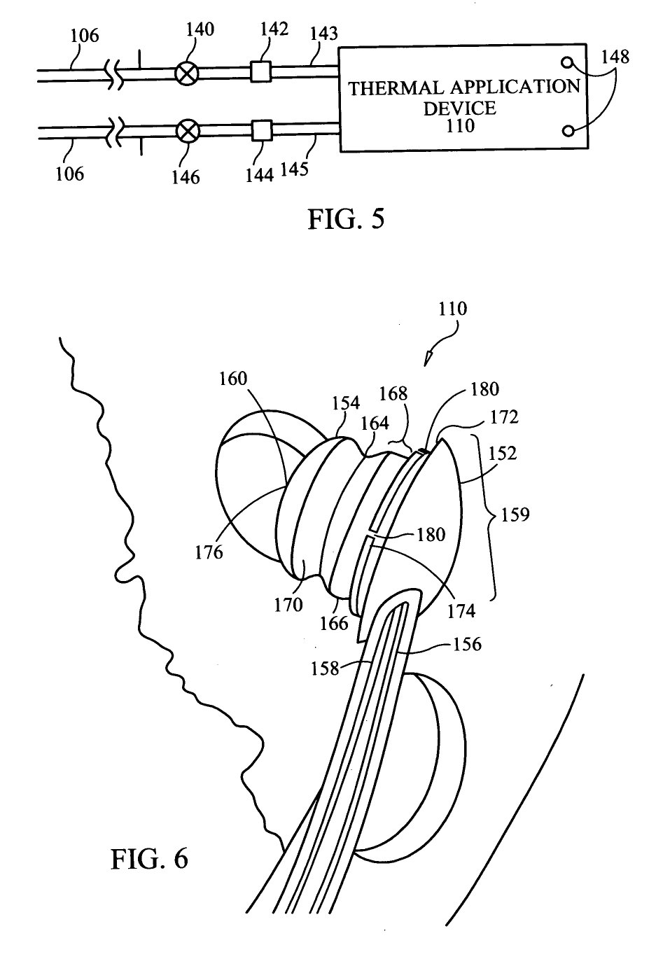

The PCU controller 114 may comprise a microprocessor, a micro-controller, an application specific integrated circuit (ASIC), and the like or alternatively, it may comprise a series of electronic circuits. The PCU controller 114 may, for example, control the speed of the pump 112, the operation of the valves 132, the control of the displays of user interface 122, and the distribution of power. A PCU Interface 120 may be provided to act as a control interface between the PCU Controller 114, the pump 112, and the valves 132. Alternatively, the PCU Interface 120 may be omitted between the PCU Controller 114, and the above-described components. The PCU controller 114 may also be interfaced with the thermal application device sensors 148, and circulation path sensors 134, e.g., one or more of the temperature or pressure sensors described herein below with respect to FIGS. 4 and 5.

The pump/controller unit 102 may include electronic circuitry that measures the fluid temperature at the inlet and outlet feed tubes 143, 145 that connect the extension tubing set 106 to the pad 110 as well as to the fluid chiller 116. In one embodiment, as illustrated in FIG. 5, thermal sensors 142 and 144 provide the temperature at the inlet 143 and outlet 145 feed tubes of the pad 110, while flow sensors 140 and 146 provide the flow rates. By measuring the difference in temperature of the thermally conductive fluid flowing between the pad inlet 143 and outlet 145 fluid pathways, and by factoring in the flow rate of the thermally conductive fluid in the pad 110, a thermal transfer efficiency function can be calculated and used as an indication to the user. The temperature sensors 142, 144 and flow sensors 140, 146 at the inlet 143 and outlet 145 of the pad 110 are preferably included as part of the fluid circulation path.

The PCU controller 114 may be interfaced with a PCU memory 126 configured to provide storage of computer software that provides the functionality of the PCU controller 114, e.g., pump 112 operation, valves 132 operation, and operation of the displays, etc. The PCU memory 126 may be implemented as a combination of volatile and non-volatile memory, such as dynamic random access memory (DRAM), EEPROM, flash memory, and the like. The PCU memory 126 may also be configured to provide storage for containing data and/or information pertaining to the manner in which PCU controller 114 may operate the pump 112, valves 132, and the displays. In one respect, the manner of operation of the above-described components may be based according to temperature measurements by temperature sensor 148.

The fluid chiller 116 may comprise any reasonably suitable type of cooling device designed to adequately cool the cooling fluid. In addition, the fluid chiller 116 may include the capability of varying the temperature of the cooling fluid. Some suitable cooling devices may include those that implement heat exchangers, heat pumps, variable capacity chillers, evaporative cooling systems, thermoelectric resistor strips and the like. The fluid chiller 116 is preferably a solid-state thermoelectric cold plate cooler (TEC) that operates on the Peltier Effect as is known in the art. By removing heat from the hot side of the plate, the reduced temperature on the cold side can be maintained. Generally, thermoelectric coolers operate by radiating heat when an electrical potential of one polarity is applied and absorbing heat when an electrical potential of the opposite polarity is applied. The thermal exchanger portion 108 of the sterile circulation path 104 is maintained in thermal contact with the cold side of the plate during operation. As the thermally conductive fluid (e.g., saline) is pumped through the thermal exchanger portion 108 of the circulation path 104, the fluid chiller 116 chills or warms the saline before it is pumped through the pad 110.

The fluid chiller controller 118 may be configured to control the operations of the fluid chiller 116. The fluid chiller controller 118 may comprise a microprocessor, a micro-controller, an application specific integrated circuit (ASIC), and the like. The fluid chiller controller 118 may include electronic circuitry to regulate the voltage to the fluid chiller 116 based on a proportional/integral feedback control algorithm. In one embodiment, feedback to control the operation of the fluid chiller controller 118 is provided via the higher temperature of two temperature sensors 148, for example thermistors, on the pad 110. When contact with the tissue is made, the thermistors 148 provide temperature indication to allow the fluid chiller controller 118 to maintain the surface at a target temperature by chilling the circulating thermally conductive fluid as required, for example to maintain brain tissue temperature at the contact point at 15° C.,±1° C. In another embodiment, feedback to control the operation of the fluid chiller controller 118 is provided via at least one temperature sensor 148, for example a thermistor, on the pad 110. In an alternate embodiment, a plurality of temperature sensors 148 provides feedback to the control operation of the fluid chiller controller 118. The system can compare the temperature measurements from the plurality of temperature sensors 148 and determine if appropriate contact with the tissue has been achieved. A significant difference among the temperature measurements would most likely indicate poor contact with the tissue surface.

Interface electronics (I/F) 124 may be provided to act as an interface between the fluid chiller controller 118 and the components for operating the fluid chiller 116, e.g., the supply of voltage to switch the polarity of the electrical potential, the control of the heat exchanger capacity, the supply of voltage to vary the speed of the compressor, etc.

The fluid chiller controller 118 may also be interfaced with a fluid chiller controller (FCC) memory 127 configured to provide storage of computer software that provides the functionality of the fluid chiller 116, e.g., heat exchanger, compressor, and the like, and may be executed by the fluid chiller controller 118. The FCC memory 127 may be implemented as a combination of volatile and non-volatile memory, such as DRAM, EEPROM, flash memory, and the like. The FCC memory 127 may also be configured to provide storage for containing data/information pertaining to the manner in which the chiller, (heat exchanger, compressor) may be manipulated in response to, for example, variations in the temperature of the cooling fluid and/or pressure in the fluid path.

A temperature sensor in the fluid chiller (not shown) is part of the pump/controller unit 102 or the fluid chiller controller 118. The pump/controller unit 102 monitors the fluid chiller temperature sensor to insure that the chiller cold plate does not cool below a selectable low temperature threshold, for example, 3° C., or warm above a selectable high temperature threshold, for example, 37° C. Should either situation result, an alarm is generated and power to the fluid chiller 116 cold plate may be disengaged.

The fluid chiller controller 118, the Interface electronics 124, the PCU controller 114 and the PCU Interface 120 can be integrated into a single controller unit (see for example the controller/conditioner of FIG. 4) or by multiple controller units as described above. The fluid chiller controller 118 may be further interfaced with the PCU controller 114. The interface may be effectuated via a wired protocol, such as IEEE 802.3, etc., wireless protocols, such as IEEE 801.11b, wireless serial connection, Bluetooth, etc., or combinations thereof.

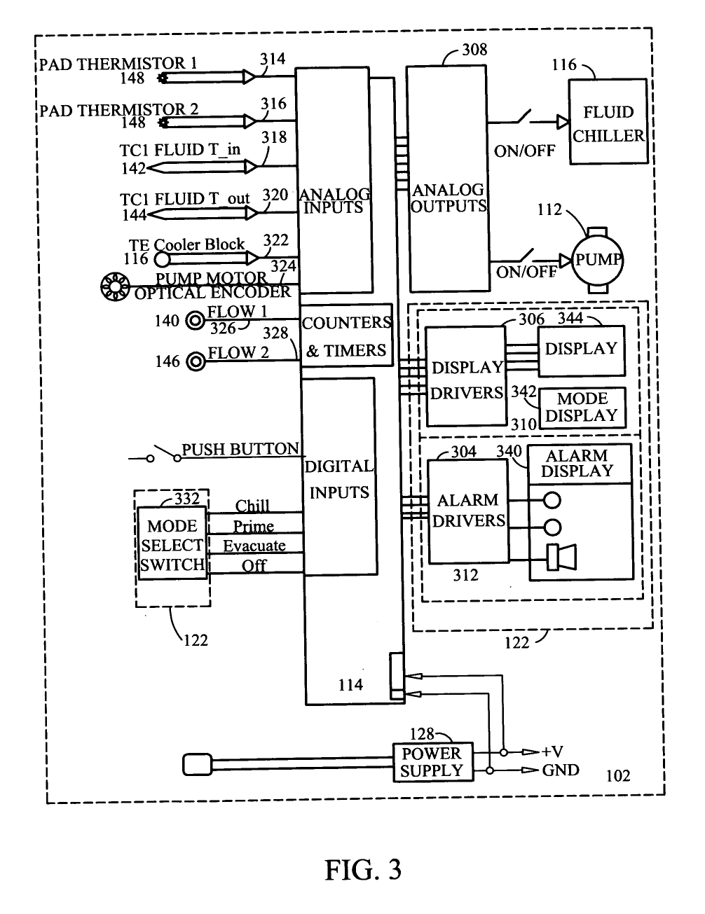

FIG. 3 is a block diagram of the pump/controller unit 102. A power supply 128 for the pump/controller unit 102 can be any power supply known in the art to power the system in a medical environment, for example a power supply that can deliver 24 VDC to power the pump 112 and fluid chiller 116, as well as other components of the pump/controller unit 102. In this embodiment, the power supply 128 employs the requisite plug and safety circuitry for use in an operating room environment as is known in the art.

The user interface 122 includes a “mode select” section 332, a “display” section 310 and an “alarm” section 312. The “display” section 310 displays an indication of the mode in which the system is operating 342, a temperature 344 measured at the underside of the pad by at least one temperature sensor, and an indication of the thermal transfer. The “alarm” section 312 includes indicators for “no flow”; “no cooling” and “no pumping” alarm conditions 340.

The PCU Controller 114 receives input signals from the various detection and measurement sensors. As indicated by the embodiment shown in FIG. 3, thermistor temperature sensors 148 provide input signals 314 and 316, thermal sensors 142 and 144 provide input signals 318 and 320, while the fluid chiller 116 temperature sensor provides input signal 322. Flow sensors 140 and 146 provide input signals 326 and 328. When the various input signals are received, the PCU Controller 114 will generate output control and display signals 308, 304, 306 for controlling the pump 112 and the fluid chiller 116, as well as the driving the alarm display 312, the temperature display 344 and the mode select display 342 of the interface unit 122. Alternatively, or in addition to, the above-described controller circuitry, a fluid chiller controller 118 may be configured to control the operations of the fluid chiller 116. The fluid chiller controller 118 may comprise a microprocessor, a micro-controller, an application specific integrated circuit (ASIC), and the like. The fluid chiller controller 118 is generally configured to manipulate the temperature of the cooling fluid by controlling the operation of the fluid chiller 116. In this regard, the fluid chiller 116 may comprise a variable speed compressor, a heat exchanger, a chilled water heat exchanger, a centrifugal chiller, and the like. More particularly, the fluid chiller controller 118 may be designed to vary the operation of one or more of the above-recited components to vary the amount of heat transfer on the refrigerant contained in the refrigeration loop of the cooling device 110 to thereby vary the cooling fluid temperature.

An optional interface electronics 124 may be provided to act as an interface between the fluid chiller controller 118 and the components for operating the fluid chiller 116, e.g., the supply of voltage to vary the speed of the compressor, control of the heat exchanger capacity, etc.

In operation, the “mode select” section 232 allows the user to choose a mode of operation. It is contemplated that the following modes of operation are included:
“OFF”—in this mode, the display is on, but neither the pump 112 nor the fluid chiller 118 is activated.
“PRIME”—turns the pump on in the forward direction, the fluid chiller 116 remains off. This mode is used during system set-up to fill the circulation path 102 and de-air the circulation path, including the pad 110.
“CHILL”—turns on both the pump 112 and the fluid chiller 116. This is the normal mode of operation for delivering chilled thermally transmissive fluid to the pad 110.
“EVACUATE”—turns off the fluid chiller 116 and operates the pump 112 in a reverse mode to cause a reverse flow of thermally transmissive fluid in the circulation path 102, thereby pumping fluid out of the pad 110 and extension tubing set 106.

FIG. 4 shows an alternate system embodiment, generally designated as 400, for applying or removing thermal energy to or from a localized region of a body tissue, while detecting potential hazards such as leaks and flow obstructions in the system. The system 400 includes a pump/controller unit 402 and a circulation path 104. Many of the components in the embodiment of system 400 correspond to the components of system embodiment 200 as described above. Accordingly, the detailed description of such components above will not be reiterated below.

The fluid circulation path 104 includes a fluid reservoir 130, an extension tubing set 106, a thermal exchanger element 108, a thermal application device 110 (referred to herein as the “pad”) for contact with the tissue to be treated, valves 416, 418 and 420, pressure sensors 422, 424 and 426 and temperature sensors 142 and 144. Additionally, an optional bubble detector 432 can be coupled to the circulation path 104. In this embodiment, the fluid reservoir 130 is preferably a saline bag, but other suitable types of fluid containers, such as but not limited to, bottles or jars may be used.

In one embodiment, three valves 416, 418 and 420 are coupled by the extension tubing set 106 to the fluid reservoir 130 and the pump 112. The valves 416, 418 and 420 may comprise any reasonably suitable type of valve designed to control the flow of a thermally conductive fluid through fluid circulation path. Some suitable valves may include those implemented in catheters, medical probes and the like. The valves 416, 418 and 420 are preferably solenoid activated pinch valves, that operate by electrical power and provide the user the capability to select an open loop or closed loop configuration for the fluid circulation path 104. In this embodiment, valve 416 is coupled between the outlet feed tube 145 of the pad 110, the outlet of valve 418 and the input of the pump 112. Valve 418 is coupled to the outlet tube of fluid reservoir 130, the outlet of valve 416 and the input of pump 112. Valve 420 is coupled between the outlet of feed tube 145 of pad 110, the inlet of valve 416 and the inlet of fluid reservoir 130. Depending on the state of each valve 416, 418 and 420 (e.g., open or closed), the thermally conductive fluid can be routed to the fluid reservoir 130 (open loop) or directly to the pump 112 (closed loop). The controller/conditioner 414 of the pump/controller unit 102 is electrically connected to the valves 416, 418 and 420 to control the state of each valve. Although there are three valves illustrated in FIG. 4, it should be understood that any number of valves might be coupled by the tubing set 106 to the fluid reservoir 130, and the pump 112.

As illustrated in FIG. 4, the controller/conditioner 414 of the pump/controller unit 102 controls the operation of the pump 112, the fluid chiller 116, a power supply (not shown) and a user interface (not shown). In this embodiment, the controller/conditioner 414 combines the operation of the PCU controller 114, a fluid chiller controller 118, the user interface 122, the optional PCU interface electronics 120, the optional PCU memory 126, the optional fluid chiller interface electronics 124 and the optional fluid chiller memory 127 into a single unit. As discussed above, the various controllers and interface electronics of the pump/controller 188, for example, the controller/conditioner 414, may be combined into a single unit or various multiple units.

Referring to system 400 of FIG. 4, the pressure sensors 422, 424 and 426 are preferably pressure transducers, but other types of sensors, such as but not limited to, optical or acoustical sensors can be used. The pressure sensors 422, 424 and 426 are coupled via the tubing set 106 to the fluid circulation path 104 and provide flow obstruction or “kink” and leak detection measurements for the system 400. The pressure sensors 422 and 426 provide P_{IN} and P_{OUT} measurements, respectively, to the controller/conditioner 414, while the pressure sensor 424 provides a P_{ALT} measurement to the controller/conditioner 414. In general, P_{IN} refers to pressure into the pad 110, P_{OUT} refers to the pressure out of the pad 110, and P_{ALT} is an alternative pressure that may measure patient pressure or internal device pressure or the like. As shown in FIG. 4, there are three sections of the fluid circulation path 102 labeled as sections “A”, “B” and “C”. Section A is defined as the portion of the path between the outlet of the pump 112 and the inlet tube 143 of the pad (e.g., P_{IN}). Section B is defined as the portion of the path between the inlet tube 143 of the pad (e.g., P_{IN}) and the outlet tube 145 of the pad (e.g., P_{OUT}). Section C is defined as the portion of the path between outlet tube 145 of the pad (e.g., P_{OUT}) and the inlet of the pump 112.

In operation, the open loop configuration is typically used during an initial system-priming mode, while the closed loop configuration is typically used to improve the thermal efficiency of the system or for leak detection when in a cooling/heating mode. When in the closed loop configuration, pressure measurements are sensitive to small losses of fluid from the system (e.g., leaks). Leaks are detected by monitoring the time derivative of the pressure, and then generating an alarm when that time derivative exceeds predetermined bounds. Additionally, when in the open or closed configuration, the pressure measurements are sensitive to flow obstructions (e.g., kinks). The relationship of the pressure sensors of FIG. 4 and the detection of flow obstructions and fluid leaks are shown in the table below (for a closed loop configuration.)

| Condition | P_{IN} | P_{OUT} | Comment |
|---|---|---|---|
| Kink A | Decrease | Decrease | |
| Leak A | Decrease | Decrease | |
| Kink B | Increase | Decrease | |
| Leak B | Decrease | Increase or Decrease | P_{OUT} may increase or |
| | | | decrease depending |
| | | | on leak size and if |
| | | | P_{OUT} is initially |
| | | | positive or negative |
| | | | gauge pressure. |
| Kink C | Increase | Increase | |
| Leak C | Decrease | Increase or Decrease | P_{OUT} may increase or |
| | | | decrease depending |
| | | | on leak size and if |
| | | | P_{OUT} is initially |
| | | | positive or negative |
| | | | gauge pressure. |
| Kink A and C | Decrease | Increase | P_{OUT} − P_{OUT} ˜ 0 |

The detection table above contains a Condition column, a P_{IN} Column, a P_{OUT} Column, and a Comment Column. When a flow obstruction (e.g., kink) occurs along Section A of the system 400, the P_{IN} and P_{OUT} will typically decrease. Similarly, when a leak occurs along Section A of the system 400, the P_{IN} and P_{OUT} will typically decrease. When a flow obstruction (e.g., kink) occurs along Section B of the system 400, the P_{IN} will typically increase, and P_{OUT} will typically decrease. Similarly, when a leak occurs along Section B of the system 400, the P_{IN} will typically increase, P_{OUT} may increase or decrease depending on the size of the leak and the initial state of P_{OUT} relative to positive or negative gauge pressure. When a flow obstruction (e.g., kink) occurs along Section C of the system 400, the P_{IN} will typically increase, and P_{OUT} will typically decrease. When a leak occurs along Section C of the system 400, the P_{IN} will typically decrease, P_{OUT} may increase or decrease depending on the initial state of P_{OUT} relative to positive or negative gauge pressure. Accordingly, flow obstructions (e.g., kinks) may be detected and isolated to specific sections of the fluid circulation path 104. In one embodiment the pressure sensors for P_{IN}, P_{ALT}, and P_{OUT} may be connected to the pad 110 via “T-Fittings” or other well known methods of connection.

System 400, as mentioned above, may operate in a closed loop or open loop configuration. The average pressure in the system is typically dictated by the flow rate provided by the pump when the system is transferred from an open loop to closed loop configuration. Once in a closed loop configuration, the difference between the P_{IN} and the P_{OUT} at any given point in time will increase as the flow rate increases; however, the average pressure will remain constant unlike in an open loop configuration. Deceasing flow rate (e.g., pump speed) prior to transferring the valves to a closed loop configuration and increasing flow after transferring to closed loop, allows for lower device pressures at higher flow rates than could be achieved in an open loop configuration, and provides a level of pressure control. Such higher flow rates can affect improved cooling efficiency. Lower device pressure minimizes risk of device mechanical failure and potentially reduces pressure exerted on the tissue. Creating a completely closed loop provides a method to control pressure in the thermal application pad 110 independent of the fluid reservoir height 130. Additionally, the valves 416, 418 and 420 provide a way to evacuate fluid from the thermal application pad 110 without the use of flow restrictive one-way valves.

An option bubble detector 432, as shown in FIG. 4, may be coupled to the fluid circulation path 104, for example between the outlet tubing 145 and the inlets of valves 416 and 420. The bubble detector 432 may be configured to indicate when air is primed from the system or when air bubbles are introduced into the fluid system. The bubble detector 432 may comprise any suitable type of bubble detector designed to indicate that bubbles are present in a fluid pathway. Some suitable bubble detectors may include those implemented in catheters, medical probes and the like. The bubble detector 432 is preferably an acoustic or ultrasonic transducer but other suitable bubble detectors, such as but not limited to, optical sensors, may be used.

As discussed above and shown in FIG. 1, a thermal application device or pad 110 is provided as part of the circulation path 104. FIG. 6 depicts an embodiment of a thermal application device 110 having an expandable body 154, for example a balloon, arranged to contact tissue to be treated via a small opening in a patient's cranium, for example a burr hole of various dimensions, such as 5 mm, 8 mm, 11 mm or 14 mm diameters. It is understood that the small opening can be a square, oval or another geometrical shape as is known in the art. The device 110 includes a cap 152, an expandable body 154, a fluid inlet conduit 182 (shown in FIG. 7) in fluid communication with fluid input lumen 156, a fluid outlet conduit 184 (shown in FIG. 7) in fluid communication with fluid output lumen 158 and at least one sensor 160. The fluid inlet conduit 182 and fluid outlet conduit 184 are in fluid communication with the interior volume 178 (not shown) of the expandable body 154. The expandable body 154 has a wall 170 that defines an interior volume 178 (not shown). The wall 170 is constructed of a resilient material that provides the ability to “deflate” or evacuate the expandable body 154. Exemplary resilient materials include rubber, silicon, flexible and thermoplastic polymers and the like.

The expandable body 154 has a proximal side 166, which is opposite the tissue contact surface area 193 (not shown here) coupled to the cap 152. The expandable body 154 is inflated and expanded by filling the interior volume 178 (not shown) of the expandable body 154 with a thermally conductive fluid circulated through lumens 156 and 158 by the pump/controller unit 102.

Additionally, as shown in FIG. 10, the expandable body 154 is provided with a physical structure that allows the expandable body 154 to be inserted through a small opening, for example a burr hole, and then deployed, thereby expanding a tissue contact surface area 193 for contacting the targeted tissue.

Further, expandable body 154 is arranged to be deployable within a region 198 between an outer barrier 196 and the tissue 197 without causing damage to tissue 197. An example of region 198 is found between the skull and the dura mater in a human. The tissue contact surface area 193 can have a shape ranging from substantially flat to concave or being flexible enough to conform to natural contours on the tissue surface. Accordingly, the device 110 provides a user (e.g., physician) with a way to thermally treat ischemic regions of the brain with a device whose geometry facilitates repeatable contact against the dura despite different skull thicknesses and dura gaps. Pleats 164 are provided in the expandable body 154 to advantageously allow the tissue contact surface area 193 of the expandable body 154 to achieve sufficient contact with the tissue to be treated so as to impart thermal change, yet also be sufficiently yielding so that the expandable body 154 does not damage the tissue.

The expandable body 154 can be made of any suitable biocompatible and/or cranial tissue compatible expandable material and is coupled to the cap 152 at its proximal end 166 to form a substantially fluid-tight seal.

The sensor 160 can be a temperature sensor or a pressure sensor for monitoring the temperature of the tissue treatment site. Alternatively, the sensor 160 can be a pressure sensor, which is used to monitor the internal pressure of the tissue being treated. The sensor 160 is coupled to a sensor connector (not shown) via wire, conduit, thermocouple, etc., to run within a sensor pathway.

FIG. 7 is a bottom view of the cap 152 of thermal transmissive device 110. The cap 152 can be made from plastic or any suitable biocompatible and/or cranial compatible material and includes a top region 159 (as shown in FIG. 6) and a bottom region 162. The cap top region 159 (as shown in FIG. 6) includes a thermally conductive fluid inlet 182 and fluid outlet 184, where the interior volume 178 of the expandable body 154 is in fluid communication with the thermally conductive fluid. The fluid inlet conduit 182 and fluid outlet conduit 184 provide a fluid path between fluid input lumen 16, fluid output lumen 158, and the interior volume 178 of the expandable body 154. The top region 159 (as shown in FIG. 6) may also include an additional auxiliary opening 186 adjacent to the fluid inlet conduit 182 and the fluid outlet conduit 184 that may be used to route sensor wires to the pump/controller unit 102.

The cap bottom region 162 includes a flange 168, which provides sufficient outer surface area for attachment of the expandable body 154, a ledge 172, which rests against the outside of the patient's cranium and limits the insertion distance of the expandable body 154 inside the patient's cranium, and a retainer 174, which provides contact with the boney structure of the cranium and exerts sufficient outward circumferential pressure to maintain contact between the patient and the device 110. The retainer 174 is arranged to be approximately the same size as the burr hole opening in the patient's cranium such that, when inserted into the patient, the retainer 174 contacts the walls of the burr hole opening. In one embodiment, the retainer 174 has protrusions 192, for example ridges or ribs (not shown) to enhance the outward circumferential pressure and secure the device 110 in position.

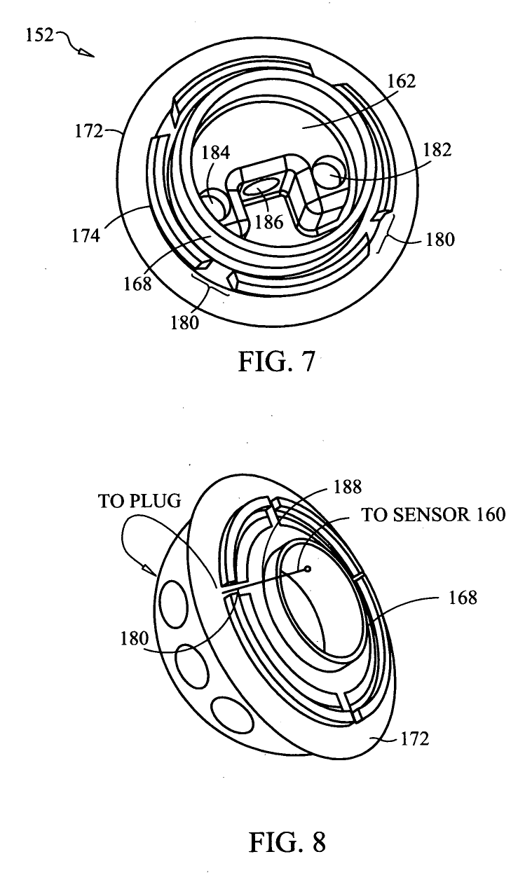

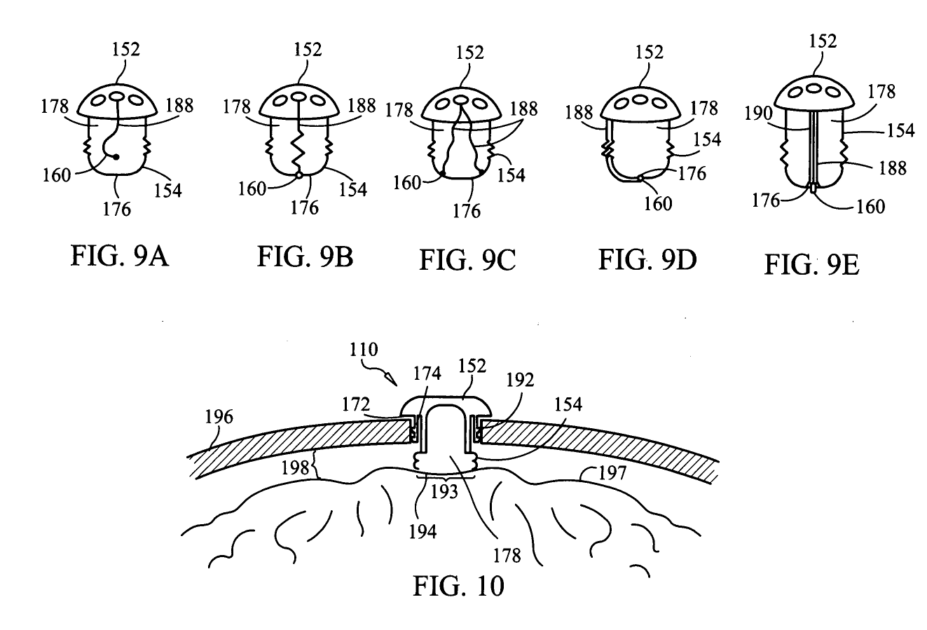

FIG. 8 is a side perspective view of the cap 152 of thermal transmissive device 110 that shows one embodiment of the routing of a sensor wire 188 to a sensor 160, for example, a thermocouple sensor (not shown). Retainer openings 180 provide a path for the sensor wire 38, for example a thermocouple wire, to connect with the sensor 160. In this embodiment, and as illustrated by FIG. 9D, the thermocouple wire 188 is attached alongside the expandable body 154 and runs to a tip portion 176 of the expandable body 154, where the thermocouple 160 resides, and provides direct contact with the targeted tissue surface 194 (not shown). The retainer openings 180 also allow the retainer 174 to achieve sufficient outward bias to provide the outward circumferential pressure described above.

As an alternative to the thermocouple routing described above, the thermocouple wire 188 can be routed through an additional auxiliary opening 186 adjacent to the fluid inlet conduit 182 and the fluid outlet conduit 184 of the cap 152. Although the thermocouple or other temperature-sensing device 160 is preferably located at the tip 176 of the expandable body 154, it may also be loosely floating in the thermally transmissive fluid as shown in FIG. 9A. In an alternative embodiment, as shown in FIG. 9C, two sensors 160 may be provided inside the expandable body 154 and attached near the tip 176. In an alternative embodiment, as shown in FIG. 9E, a flexible tube 190 provides a sheath or enclosure for the sensor or pigtail wire 188 in order to insulate the temperature sensor 160, for example a thermistor, from the cooling fluid. The flexible tube 190 can be potted at its proximal and distal ends to provide a watertight seal.

In practice, the expandable body 154 is inserted into the body of a subject to be treated. When the expandable body 154 is positioned at a desired treatment region, fluid is introduced into the expandable body 154 through fluid inlet 182, thereby flowing into the interior volume 178 of expandable body 154 and thereby “deploying” the expandable body 154. Referring to FIG. 10, when the expandable body 154 is in its deployed state, the fluid continues to flow through the circuit and thereby thermally affects the tissue contact surface area 193 of expandable body 154, which thereby thermally affects the tissue treatment site 194 of the brain tissue 197. The tissue treatment site 194 may be identified via a variety of methods for mapping the centers of brain functions. The protrusions 192 of the retainer 174 assist in securing the device 110 to the skull 196.

Additionally, the above described system and device can be used in other parts of the body in instances where local tissue temperature needs to be controlled or modulated. In such instances, thermal therapy may involve either chilled or heated fluid inside the expandable body to achieve the desired result. For example, the system and device could be applied to organs prior to or post transplant (e.g. kidney) to minimize ischemia and swelling. Further, the system and device could use be used to minimize uterine irritability in a female subject that is at risk for premature delivery.

In a method of use, the device 110 is inserted into the body of a subject to be treated and is positioned against the desired tissue treatment site 194, such that the tissue contact surface area 193 of expandable body 154 is in thermal communication with the tissue treatment site 194. A thermally-transmissive fluid is introduced into the device 110 via the fluid inlet conduit 32. The fluid travels along the fluid path to the contact surface area 193 and exits the device 110 via the fluid outlet conduit 184. The fluid continues to flow through the device 110, thereby thermally affecting the tissue treatment site 194. As described above, the fluid inlet conduit 182 and fluid outlet conduit 184 are coupled to the fluid input lumen 156 and fluid output lumen 158, which are in turn coupled to a fluid inlet tube 143 and fluid outlet tube 145, respectively of the fluid path 102. The fluid inlet tube 143 and fluid outlet tube 145 are coupled to a control device/pump assembly that provides a fluid circulation circuit to pump and control the thermal fluid through the device 110.

In an exemplary embodiment, the expandable body 154 is infused with a low-pressure thermally conductive fluid to expand its shape to a deployed state, the expansion causing contact with the tissue to be treated. The fluid can thereby impart a thermal change to the expandable body 154 that in turn imparts a thermal change to the contacted tissue. For example, the expandable body 154 can be deployed with a thermally conductive fluid having a pressure of between about 0 psi and 5 psi.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. A system for thermally affecting tissue of a patient, comprising:
a pump/controller unit having;
a pump for pumping a thermally conductive fluid through the system;
a fluid chiller for thermally treating the conductive fluid;
a controller circuit for measuring and controlling the temperature of the conductive fluid; and
a fluid circulation path having;
an extension tubing set for circulating the thermally conductive fluid;
a thermal application device defining at least one flow passage therein and in fluid communication with the tubing set; and
a thermal exchanger element in fluid communication with the tubing set, wherein the thermal exchanger element is configured to interface with the fluid chiller;
wherein the tubing set operably interfaces with the pump to enable the pump to circulate the thermally conductive fluid through the fluid circulation path.

2. The system of claim 1 further comprising:
a fluid reservoir in fluid communication with the tubing set; and
at least three valves in fluid communication with the tubing set and the fluid reservoir.

3. The system of claim 1, wherein the pump is a peristaltic pump.

4. The system of claim 1, wherein the tubing set is a flexible tube.

5. The system of claim 1, wherein the fluid chiller is a thermoelectric cooler.

6. The system of claim 1, wherein the thermal exchanger element has its inlet port and outlet port on one end.

7. The system of claim 1, wherein the thermal exchanger element has its inlet port and outlet port on opposing ends.

8. The system of claim 1, wherein the fluid circulation path further comprises at least one temperature sensor proximate and in fluid communication with a fluid inlet conduit of the thermal application device.

9. The system of claim 1, wherein the fluid circulation path further comprises at least one temperature sensor proximate and in fluid communication with a fluid outlet conduit of the thermal application device.

10. The system of claim 1, wherein the fluid circulation path further comprises at least one flow sensor proximate and in fluid communication with a fluid outlet conduit of the thermal application device.

11. The system of claim 1, wherein the fluid circulation path further comprises at least one flow sensor proximate and in fluid communication with a fluid outlet conduit of the thermal application device.

12. The system of claim 1, wherein the thermal application device includes an expandable body defining a tissue contact area and at least one temperature sensor located within the expandable body.

13. The system of claim 12, wherein the at least one temperature sensor is located proximate a tip of the expandable body.

14. The system of claim 1, wherein the thermal application device includes an expandable body defining a tissue contact area and at least one pressure sensor located within the expandable body.

15. The system of claim 14, wherein at least one pressure sensor is located proximate a tissue contact area of the expandable body.

16. The system of claim 1, wherein the controller circuit comprises a controller portion, an interface portion, and a user interface;
wherein the controller portion communications with the interface portion to provide control and monitoring of the pump and a user interface display.

17. The system of claim 1, wherein the thermal application device includes an expandable body defining a tissue contact area and at least one temperature sensor located proximate a tissue contact area of the expandable body for measuring temperature at the tissue of the patient.

18. A system for thermally affecting tissue of a patient, comprising:
a pump/controller unit having;
a pump for pumping a thermally conductive fluid through the system;
a fluid chiller for thermally treating the conductive fluid;
a controller circuit for measuring and controlling the temperature of the conductive fluid; and
a fluid circulation path having;
an extension tubing set for circulating the thermally conductive fluid;
a thermal application device defining at least one flow passage therein and in fluid communication with the tubing set, wherein the thermal application device includes an expandable body defining a tissue contact area and at least one temperature sensor located within the expandable body; and
a thermal exchanger element in fluid communication with the tubing set, wherein the thermal exchanger element is configured to interface with the fluid chiller;
wherein the tubing set operably interfaces with the pump to enable the pump to circulate the thermally conductive fluid through the fluid circulation path;
wherein the fluid circulation path includes at least one temperature sensor proximate and in fluid communication with a fluid conduit of the thermal application device.

19. A system for thermally affecting tissue of a patient, comprising:
a pump/controller unit having;
a pump for pumping a thermally conductive fluid through the system;
a fluid chiller for thermally treating the conductive fluid;
a controller circuit for measuring and controlling the temperature of the conductive fluid; and
a fluid circulation path having;
an extension tubing set for circulating the thermally conductive fluid;
a thermal application device defining at least one flow passage therein and in fluid communication with the tubing set, wherein the thermal application device includes an expandable body defining a tissue contact area and at least one temperature sensor located within the expandable body;
a fluid reservoir in fluid communication with the tubing set;
at least three valves in fluid communication with the tubing set and the fluid reservoir; and
a thermal exchanger element in fluid communication with the tubing set, wherein the thermal exchanger element is configured to interface with the fluid chiller;
wherein the tubing set operably interfaces with the pump to enable the pump to circulate the thermally conductive fluid through the fluid circulation path.

20. A method of thermally affecting a tissue treatment site in the body of a patient, the method comprising:
selecting a medical device to thermally affect the tissue treatment site, the medical device including an expandable body defining a tissue contact area;
creating an opening in the patient's body;
inserting the expandable body into the opening such that the tissue contact area is in thermal communication with the tissue treatment site; and
infusing a thermally transmissive fluid into the expandable body.

21. The method according to claim 20, further comprising selecting a tissue treatment site, the tissue treatment site being in the patient's head.

22. The method according to claim 20, further comprising sensing temperatures from at least one point in the tissue contact area.

23. The method according to claim 20, further comprising controlling the temperature of the thermally transmissive fluid in responsive to the temperatures sensed from the at least one point in the tissue contact area.

24. The method according to claim 20, wherein the tissue treatment site is selected based on a previous mapping of centers of brain function.

25. The method according to claim 20, wherein the infused thermally conductive fluid is a chilled saline fluid.

26. A medical device for thermally affecting tissue, comprising:
a cap including a bottom region and a top region, the top region containing a fluid inlet conduit and a fluid outlet conduit; the cap being securable to an opening in the patient; and
an expandable body including a wall defining an interior volume and a tissue contact surface, the expandable body being coupled to the cap bottom region such that the interior volume is in fluid communication with the fluid inlet and the fluid outlet.

27. The device for thermally affecting tissue in a patient according to claim 26, further comprising at least one temperature sensor proximate the tissue contact area of the expandable body for measuring temperature at the tissue of the patient.

28. The device for thermally affecting tissue in a patient according to claim 27, wherein the at least one temperature sensor is enclosed within the wall of the expandable body.

29. The device for thermally affecting tissue in a patient according to claim 26, further comprising at least one pressure sensor proximate the tissue contact area of the expandable body for measuring temperature of the tissue of the patient.

30. The device for thermally affecting tissue in a patient according to claim 29, wherein the at least one pressure sensor is enclosed within the wall of the expandable body.

31. The device for thermally affecting tissue in a patient according to claim 26, further comprising at least one pressure sensor proximate the tissue contact area of the expandable body for measuring pressure of the tissue of the patient.

32. The device for thermally affecting tissue in a patient according to claim 31, wherein the at least one pressure sensor is enclosed within the wall of the expandable body.

33. The device for thermally affecting tissue in a patient according to claim 26, wherein the expandable body is a balloon.

34. The device for thermally affecting tissue in a patient according to claim 26, wherein the expandable body has pleats.

35. The device for thermally affecting tissue in a patient according to claim 26, wherein the tissue contact surface is substantially concave.

36. The device for thermally affecting tissue in a patient according to claim 26, wherein the tissue contact surface is substantially flat.

37. The device for thermally affecting tissue in a patient according to claim 26, wherein the tissue contact surface substantially conforms to the tissue being treated.

38. The device for thermally affecting tissue in a patient according to claim 26, wherein the expandable body is made from a resilient material.

39. The device for thermally affecting tissue in a patient according to claim 26, further comprising a low pressure thermally conductive fluid control system operably connected to the cap fluid inlet and the cap fluid outlet.

40. A medical device for thermally affecting tissue, comprising:
a cap including a bottom region and a top region, the top region containing a fluid inlet conduit and a fluid outlet conduit; the cap being securable to an opening in the patient;
an expandable body including a wall defining an interior volume and a tissue contact surface, the expandable body being coupled to the cap bottom region such that the interior volume is in fluid communication with the fluid inlet and the fluid outlet; and
at least one temperature sensor proximate the tissue contact area of the expandable body for measuring temperature at the tissue of the patient;
wherein the tissue contact surface substantially conforms to the tissue being treated.

41. The device for thermally affecting tissue in a patient according to claim 40; wherein the expandable body is made from resilient material.

42. A medical device for thermally affecting tissue, comprising:
a cap including a bottom region and a top region, the top region containing a fluid inlet conduit and a fluid outlet conduit; the cap being securable to an opening in the patient;
an expandable body including a wall defining an interior volume and a tissue contact surface, the expandable body being coupled to the cap bottom region such that the interior volume is in fluid communication with the fluid inlet and the fluid outlet; and
at least one pressure sensor proximate the tissue contact area of the expandable body for measuring pressure at the tissue of the patient;
wherein the tissue contact surface substantially conforms to the tissue being treated.

43. The device for thermally affecting tissue in a patient according to claim 42; wherein the expandable body is made from resilient material.

44. A method of using an expandable element to affect a thermal energy change in tissue of a patient's body, comprising:
creating a small opening in the patient's skull;
securing a cap within the small opening, such that the expandable body is positioned between the boney structure and the tissue to be treated; and
infusing a low-pressure thermally conductive fluid into the expandable element.

45. The system of claim 1, wherein the fluid circulation path further comprises a leak detection element.

46. The system of claim 45, wherein the leak detection element includes a plurality of pressure sensors in communication with the thermal application device to provide leak detection for the fluid circulation path.
